(19) United States
(12) Patent Application Publication
(10) Pub. No.: US 2004/0158283 A1
(43) Pub. Date: Aug. 12, 2004

(54) Understocking with sleeve for positioning a gel pad

(76) Inventors: C. David Shook, Mansfield, OH (US); David J. Hoy, Mansfield, OH (US)

Correspondence Address: Woodling, Krost and Rust (Kenneth L. Mitchell), 9213 Chillicothe Road, Kirtland, OH, 44094, US

(21) Appl. No.: 10/603,643

(22) Filed: Jun. 25, 2003

Related U.S. Application Data
(60) Provisional application No. 60/445,549, filed on Feb. 6, 2003.

(51) Int. Cl. A61B 17/12

(52) U.S. Cl. 606/201; 606/204; 601/151

(57) ABSTRACT

The invention is a gel pad which is placed in a pocket or sleeve in an understocking. The understocking can be a compression stocking and the stocking may have a plurality of pockets or sleeves in which the gel pad(s) may reside depending on the treatment plan of the doctor or the clinician. The sleeve enables positioning of the pad where treatment is needed Pressure is applied to the affected area without having the pad in contact with the area or its dressing.

This application claims priority of previously filed application serial No. 60/445,549 filed Feb. 6, 2003.

### FIELD OF THE INVENTION

The invention is in the field of devices used to treat venous insufficiencies of the lower extremities.

### BACKGROUND OF THE INVENTION

A good description of the problems and issues confronting patients with venous insufficiencies and other maladies is found in U.S. Pat. No. 5,823,195, issued Oct. 28, 1998 which is incorporated herein by reference hereto.

U.S. Pat. No. 5,092,347 to Shaffer et al. is directed to a personalized sock kit for relieving foot and ankle pain. Shaffer et al. describes an orthotic device which relieves the pain caused by deformities of the foot by using a plurality of specific paddings that self adhere to specific mapped out areas on a unique sock.

Chronic venous insufficiency is a common problem related to complications of deep venous thrombosis, varicose veins and other causes of incompetent valves in the veins of the lower extremities. Chronic venous insufficiency results in venous hypertension especially in the ankle area causing chronic swelling, thickening of the skin and bronze discoloration of the skin. Ultimately skin breakdown and ulceration develops. Ulcerations and skin breakdown occur in different places from person to person and therefore the positioning of the pads becomes important on a patient by patient basis.

Graded compression stockings have been a major help in preventing the swelling and other complications of chronic venous insufficiency. Graded compression stockings have the highest level of compression at the ankle and it decreases as the stocking goes up the leg. It is important to fit these stockings properly and accurately in order to get the best results.

As good as these stockings are, many people still develop ulcers around the ankle. This is because the boney prominences and depressions around the ankle cause a nonuniform surface. The compression therefore is not equal and the areas of lesser compression are vulnerable to ulcer formation.

To solve this problem, we have devised gel pads that fit within an understocking in the ankle area and exert a more uniform pressure than the understocking alone is able to do. The goal is to neutralize the elevated internal venous pressure by exerting an equal external compression force. If this can be done, the swelling, bronze discoloration and ulcerations can be avoided. To do this, the depressions had to be filled in. Since the pad is within a sleeve in the understocking it does not directly contact the affected area or the bandaging of the affected area.

The invention will be better understood when reference is made to the SUMMARY OF THE INVENTION, BRIEF DESCRIPTION OF THE DRAWINGS, DESCRIPTION OF THE INVENTION and claims which follow hereinbelow.

### SUMMARY OF THE INVENTION

The invention is a gel pad which is placed in a pocket or sleeve in an understocking. The understocking can be a compression stocking and the stocking may have a plurality of pockets or sleeves in which the gel pad(s) may reside depending on the treatment plan of the doctor or the clinician.

Alternatively, the understocking may have a sleeve which extends around most of the stocking. This enables the pad to be placed where it is desired, i.e., over the wound area. Pads of any size or shape may be used in any location as desired.

After several prototype designs, we developed ankle pads in rectangular configurations made out of a gel material. This gel molds under the understocking to fill in the depressions around the malleolus. It is placed low in the ankle and extends well above the malloelus. The reason it goes up the leg so far is to cover the lowest one or two perforating veins in the medial ankle. Superficial veins are generally near the surface and communicate with deep veins which are further from the surface. Perforating veins connect the superficial veins to the deep veins.

The pad is 8 centimeters in width and 15 centimeters long and 5 millimeters thick. The gel pad is covered with a thin clear plastic skin that is very soft and pliable. The corners of the pad are rounded. The plastic skin is sealed with an ultrasound seal. The pad is firm enough that it holds its shape well. It does however at body temperature under the stocking mold somewhat to the shape of the ankle. Even though 5 millimeters seems very thin, it is enough to increase the pressure at the ankle level even in the areas of depression. It therefore gives a more even distribution to the pressure under the understocking at the ankle level. The pad can be used on the medial or lateral aspects of the ankle alone or on both sides simultaneously.

The plastic skin, which covers the pad, is a soft non-breathable material that cannot be placed next to the skin. It was therefore necessary to devise a vehicle to keep the pad in place, protect the skin from the pad, and make it possible to pull the heavy compression stocking over the understocking with the gel pad in place.

Our solution to this problem was to devise a closed-toe sock or understocking that extends up the leg to a level just above the length of the pad. The material is a stretchy material that holds the pads in place well. It is a double-thickness, except for the toe, so that the pad can fit between the two layers of the sock or understocking. There is a window at the upper end of the understocking both medial and lateral through which the pads are placed into position. The sock understocking is applied first (i.e., the stocking is put on) and once in place, the pads are inserted through the windows into position. The elasticity of the understocking is enough to keep the pads in position while the compression stocking is pulled over it. Once the compression stocking is pulled over the pads and into its normal position, the system stays in place until the understocking is removed.

If there are open ulcers present, local dressings are applied under the sock (understocking) first. The pad is then inserted and followed by the compression stocking. This system can be used to enhance and speed up the healing of open ulcers. It is also very effective in maintaining healing once the ulcers are healed. The pad alone can be incorporated into an Unna boot to increase the effectiveness of the Unna boot. The increased even pressure around the ankle squeezes out fluid from the soft tissue under the pad and hastens healing.

It is an object of the invention to provide a delivery mechanism for the application of pressure in certain locations on a person's (patient's) leg, ankle or foot.

It is an object of the invention to provide an understocking having a pocket enabling a pressure pad to be positioned therein and placed where needed.

It is an object of the invention to provide an understocking which fits a variety of patients and which includes a pocket or pockets therein enabling placement of a malleable pad or pads within the pocket to treat affected portions of a leg or ankle.

It is an object of the invention to provide a stocking which enable use of a pad under compression but which separates the pad from the skin or ulceration which the pad is treating.

It is an object of the invention to provide an understocking which allows variation as to the position in which the pad is placed.

It is an object of the invention to provide an understocking or sock which may be used in conjunction with a graded compression stocking.

It is an object of the invention to provide an understocking which allows for use of pads of different shapes and thicknesses.

These and additional objects of the invention will become apparent when reference is made to the Brief Description of the Invention, Detailed Description of the Invention and claims which follow hereinbelow.

### BRIEF DESCRIPTION OF THE DRAWINGS

FIG. 1 is a top view of a gel pad.

FIG. 2 is a side view of a gel pad.

FIG. 3 is a cross-sectional view of a gel pad.

FIG. 4 is a view of an understocking illustrating a sleeve in which the gel pad resides.

FIG. 5 is a side view of the understocking of FIG. 4.

FIG. 6 is a view of an understocking similar to FIG. 4.

FIG. 7 is a front view of an understocking illustrating the sleeve in which the gel pad resides.

FIG. 8 is a view similar to FIG. 4 illustrating a gel pad in a sleeve.

FIG. 9 is a view of the understocking with a gel pad placed on a person's foot at or near the bony prominence of the ankle.

FIG. 10 is a view of the understocking with the gel pad placed on a person's foot and with a compression stocking further used.

FIGS. 11, 12 and 13 are similar to FIGS. 8, 9 and 10 and illustrate the understocking and a smaller pad in a different position on the same foot or in approximately the same position on a different foot.

A better understanding of the invention will be had when reference is made to the Description of the Invention and claims which follow hereinbelow.

### DESCRIPTION OF THE INVENTION

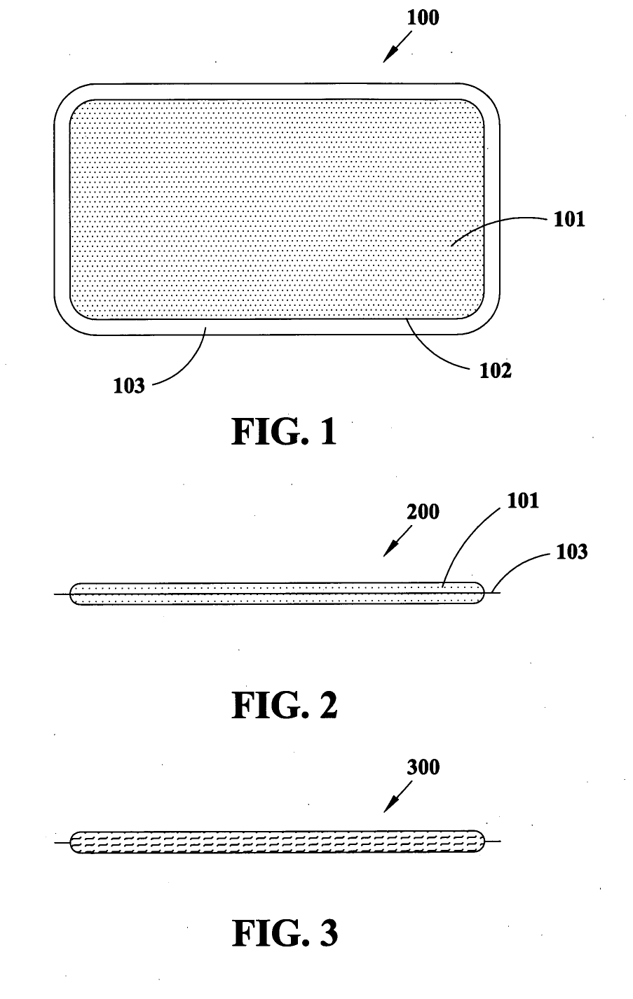

FIG. 1 is a top view 100 of a gel pad 101 which is sealed in a flexible and pliable plastic. The plastic may be any flexible plastic and preferably it is transparent. The gel can be any amorphous material. Reference numeral 102 represents the seal which retains the gel and reference numeral 103 is the plastic seal border which surrounds the gel pad. FIG. 2 is a side view 200 of a gel pad 101 which further illustrates the plastic seal border 103. FIG. 3 is a cross-sectional view 300 of a gel pad 101. The pad as illustrated is 8 centimeters in width and 15 centimeters long and 5 millimeters thick. However, the pad may be any shape and thickness as long as the pressure stocking (i.e, a compression stocking) or pressure gradient stocking (i.e., a pressure gradient compression stocking) may be fit over the understocking positioning the pad(s).

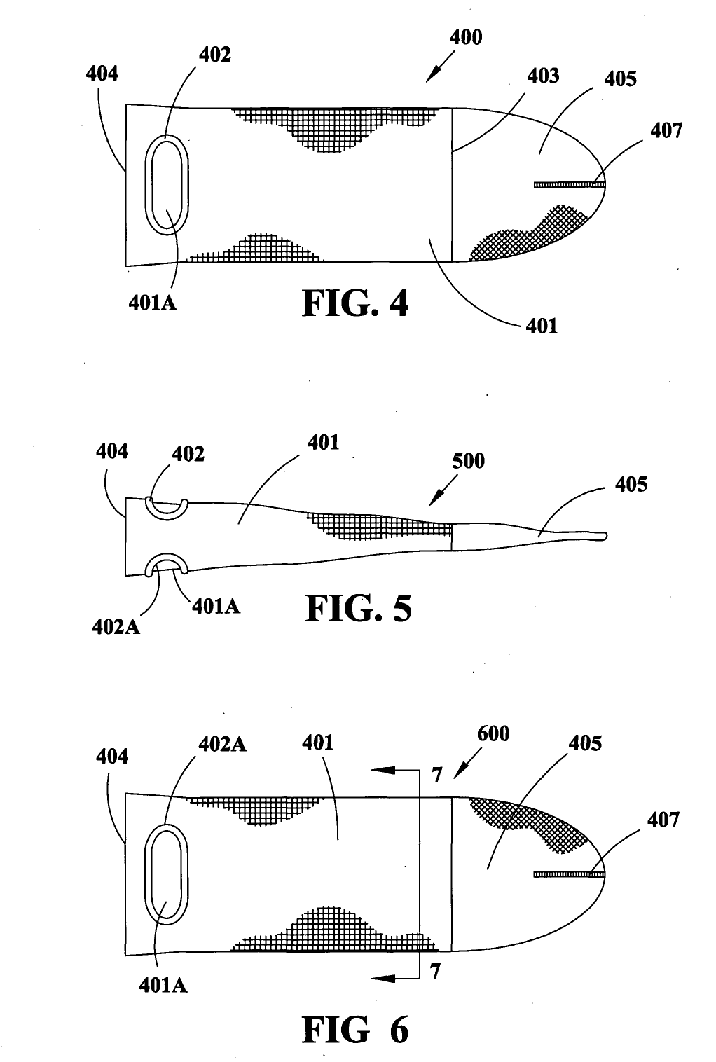

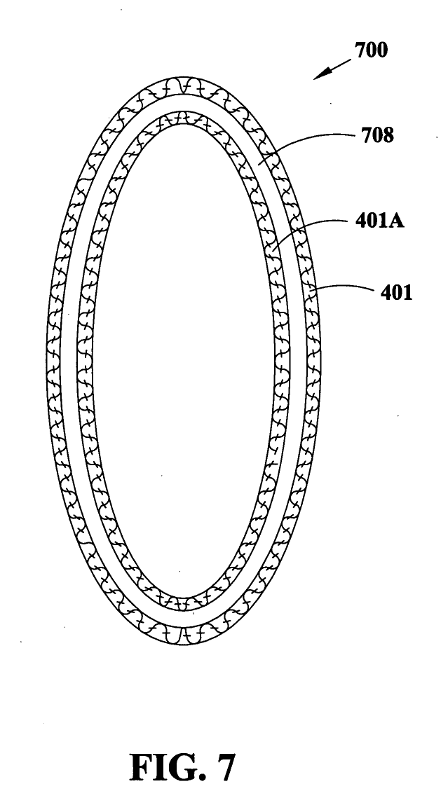

FIG. 4 is a view of an understocking 400 illustrating an opening 402 to a sleeve 708 which extends around most of the stocking. Sleeve 708 can best be seen in FIG. 7 and extends 360 degrees around the understocking. Sleeve 708 is defined by sock surfaces 401/401A. The understocking may optionally be a compression understocking or a graded compression stocking meaning that it applies more pressure at its bottom than top along a pressure gradient. This would enable use of the understocking alone for some patients.

It is for the treating doctor and clinician to decide whether or not the understocking should be a compression understocking. Further, it for the treating doctor and clinician to decide whether or not a compression stocking should be used over the understocking and the amount and type of compression to be applied.

Referring again to FIG. 4, the sleeve is formed by an outer portion 401 and an inner portion 401A. Both the inner portion 401 and the outer portion 401 are woven as is the toe portion 405. The understocking can be made from natural and/or synthetic fibers. Nylon, polyester and cotton are examples of the materials which may be employed in the manufacture of the understocking. The understocking has sufficient elasticity to secure the gel pad 101 in place. The compression stocking (outer stocking) or gradient compression stocking fits over the understocking 400 and secures the understocking and pads to the user's (patient's) body. The sleeve is bounded by stitching at line 403 and the end of the understocking 404 which is folded over itself. Referring to FIG. 7, sleeve 708 is best illustrated as a space bounded by the inner portion 401 of the understocking and the outer portion 401A of the understocking.

Sleeve 708 does not extend to toe portion 405 which is defined by stitching 403. Stitching 403 secures inner portion 401A to the outer portion 401. Stitching 403 terminates the sleeve 708. The sleeve 708 is large and provides considerable room for positioning a pad or pads over affected areas of a patient's ankle, foot and lower leg. End of understocking 404 is a portion of the understocking which is folded over and onto itself to form the portions 401/401A thereof. Stitching 407 terminates toe portion 405 of the understocking.

The instant invention may be used in a preemptive manner such that the treating doctor or clinician may apply pressure to affected areas of discoloration so as to prevent ulcerations.

The understocking 400 is flexible and can be stretched to fit the user. Different sizes of the understocking fit users in different groups. Line 403 indicates the extent of the sleeve 708 and the beginning of the toe portion. Gel pad 101 can be placed at any location within the sleeve.

It is specifically contemplated that various chambers, pockets or sleeves may be incorporated in sleeve 708 by stitching or otherwise partitioning sections thereof. FIG. 5 is a side view 500 of the understocking of FIG. 4 illustrating openings 402 and 402A. These openings or windows 402/402A may be stitched to maintain the integrity of the sock or understocking 400 or them may be formed using another border material. Two openings enable easy access to the entire sleeve 708. One window or opening may be sufficient in certain circumstances.

These openings or windows can be used to insert, by hand, a gel pad such as pad 101 into sleeve 708 to position it anywhere it is desired and needed. Sleeve 708 is illustrated in FIG. 7. FIG. 6 is a view 600 of an understocking similar to FIG. 4 illustrating opening 402A.

FIG. 7 is a cross-sectional view 700 taken along the lines 7-7 of FIG. 6 illustrating sleeve or space 708 formed by and having outer portion 401 and inner portion 401A spaced apart therefrom. Sleeve 708 extends circumferentially around the entire understocking.

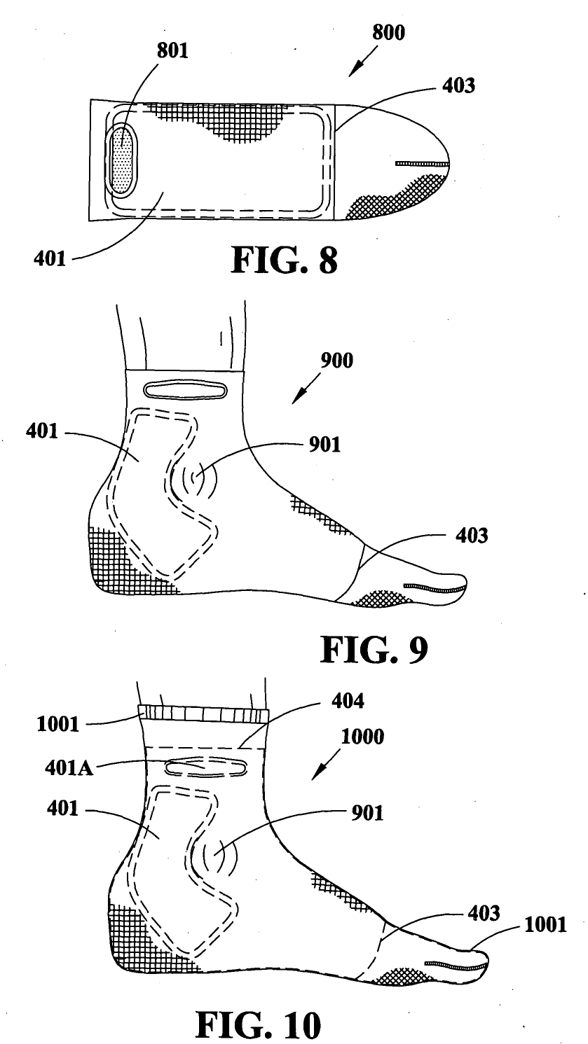

FIG. 8 is a view 800 similar to FIG. 4 illustrating a large gel pad 801 in sleeve 708 of understocking 400. In FIG. 8, gel pad 801 is large and illustrates sleeve 708 being almost totally occupied on one side of the understocking down to stitching 403. Although this is not the ordinary usage of a pad, it does demonstrate the extent to which a pad could be used. However, for the purposes of treating venous insufficiency it has been determined that use of smaller pads in specific locations is best.

The instant invention is advantageous in that the pads need not be applied directly to the skin or dressing at the location of an ulceration. Rather, a layer of cloth (synthetic or natural) 401A is placed between the pad and the user's skin. The understocking is pulled up as a person would normally put on a stocking. The pad is then positioned within sleeve 708 in proximity to the area of the foot, ankle or leg to be treated. Alternatively, the pad may be placed in the sleeve of the understocking first and then the understocking with the pad therein may be applied to a person's foot, ankle and lower leg. Using this method, however, may necessitate adjustment of the pad after the understocking has been put on the user. Next, either a compression stocking or a pressure gradient stocking will be applied over the understocking which keeps the pad in place and provides pressure to the affected limb as desired.

FIG. 9 is a view 900 of the understocking with a gel pad 101 positioned on a person's foot behind the ankle 901. FIG. 10 is a view 1000 of the understocking of the invention with a gel pad placed therein can been seen in phantom positioned on a person's foot with compression stocking 1001 employed over the entire understocking. The compression stocking 1001 may be a pressure gradient stocking which applies higher pressure at the bottom of the limb than at the top of the limb. Referring again to FIG. 10, compression stocking 1001 obscures the understocking of the invention. In FIG. 10, the sleeve bounded by reference numerals 404, 403 as well as the mouth 402 of the sleeve can be seen in phantom. The compression stocking resides completely over the understocking which positions the gel pad.

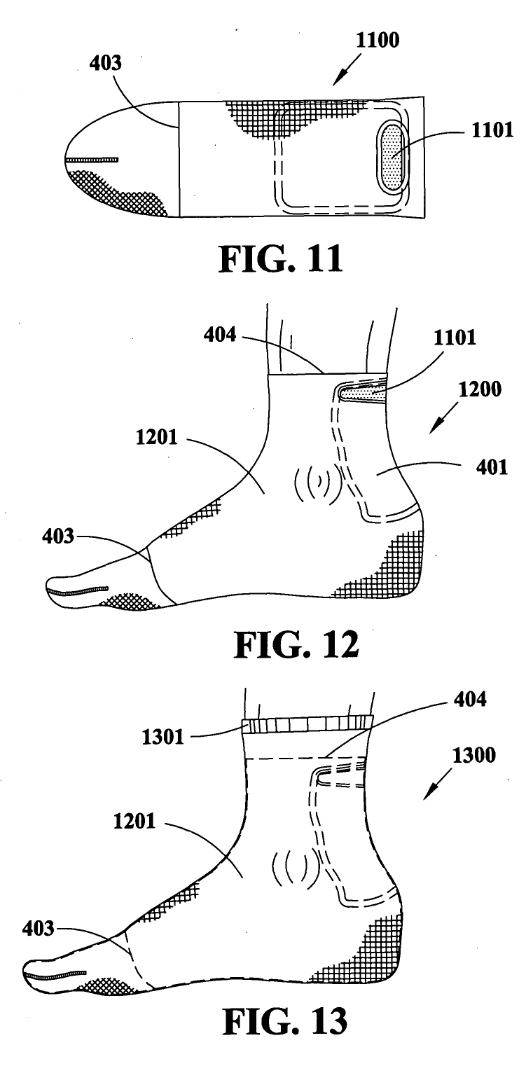

FIGS. 11, 12 and 13 are views 1100, 1200 and 1300 which illustrate the understocking and a smaller pad 1101 in a different position on a foot of a patient. The smaller pad 1101 is placed by way of example behind the ankle and above it as illustrated in FIG. 12. Reference numeral 1201 indicates the understocking of the invention in FIGS. 12 and 13. The pad can be any size (length, width and thickness) and it can be placed anywhere in the sleeve which extends from lines 404 to 403. Reference numeral 1300 is a view used to signify a compression stocking 1301 which is placed completely over the foot, ankle and lower leg of the user.

It has been our experience that not only does this system speed up the healing of open ulcers, but it maintains healing. To our surprise, the skin and soft tissue and areas of chronic scarring become soft and pliable over a period of time, the induration disappears, bronze discoloration slowly fades. This system allows people to return to work and keeps them out of the ulcer clinic.

If open ulcers are present, the sock alone is used at night to keep the dressing in place. In the morning, the pads are inserted and the compression stocking is applied. Daily bathing is possible in this type of treatment.

The compression stocking that works best with this system is the Class II (30-40 mm) stocking. In the older patients, it may be utilized with lesser degrees of compression even with the TED type stockings, since high compression may become problematic.

This mode of treatment is versatile, and allows people with severe chronic venous insufficiency to remain ambulatory. It also maintains healing of previous ulcerated areas and even allows people to return to work.

The patient may have bandages over the ulceration. The inner portion of the stocking material engages the bandages over the ulceration and/or the skin of the patient. The gel pad resides in contact with the inner and outer portions of the understocking but does not contact the skin or bandages of the patient. A compression stocking is then placed over the outer portion of the stocking and functions to, among other things, keep the gel pad in position.

While the invention has been set forth by way of example herein those skilled in the art will readily recognize that changes and/or modifications may be made to the invention without departing from the spirit and scope of the appended claims.

1. A stocking comprising a sleeve and a gel pad within said sleeve.

2. A stocking as claimed in claim 1 further comprising an opening in said sleeve for accessing, placing and positioning said gel pad within said sleeve.

3. A compression stocking comprising a sleeve and a gel pad within said sleeve.

4. A compression stocking as claimed in claim 3 further comprising an opening in said sleeve for accessing, placing and positioning said gel pad within said sleeve.

5. A stocking as claimed in claim 2 wherein said stocking includes a folded portion and a stitched portion to form said sleeve.

6. A compression stocking as claimed in claim 4 wherein said compression stocking includes a folded portion and a stitched portion to form said sleeve.

7. A stocking as claimed in claim 5 wherein said sleeve extends 360 degrees within said stocking.

8. A compression stocking as claimed in claim 6 wherein said sleeve extends 360 degrees withing said stocking.

9. A device for treating venous insufficiency comprising an understocking having a sleeve and a gel pad within said sleeve.

10. A device for treating venous insufficiency as claimed in claim 9 further comprising an opening in said sleeve for accessing, placing and positioning said gel pad within said sleeve.

11. A device for treating venous insufficiency as claimed in claim 10 further comprising a compression stocking residing over said understocking.

12. A device for treating venous insufficiency as claimed in claim 9 further comprising a compression stocking residing over said understocking.

13. A device for treating venous insufficiency as claimed in claim 9 wherein said understocking includes a folded portion and a stitched portion forming said sleeve.

14. A device for treating venous insufficiency as claimed in claim 13 wherein said understocking is made from a material selected from the group of nylon, polyester, and cotton.

15. A device for treating venous insufficiency as claimed in claim 13 wherein said understocking is made from a material selected from an elastic material.

16. A method of treating a patient having venous insufficiency comprising the steps of:
applying an understocking having a sleeve onto the foot, ankle and leg of said patient;
inserting a gel pad into said sleeve; and,
positioning said gel paid about said foot, ankle and leg of said patient to apply pressure to reduce said venous insufficiency.

17. A method of treating a patient having venous insufficiency as claimed in claim 16 further comprising the steps of:
applying a compression stocking over said understocking and pads.

18. A method of treating a patient having venous insufficiency as claimed in claim 16 wherein said understocking is a compression stocking.

19. A method of treating a patient having venous insufficiency as claimed in claim 17 wherein said understocking is a compression stocking.

20. A method of treating a patient having venous insufficiency as claimed in claim 16 wherein said understocking is a compression stocking.
